(19) United States
(12) Patent Application Publication
(10) Pub. No.: US 2025/0277090 A1
(43) Pub. Date: Sep. 4, 2025

(54) WATER-DISSOLVABLE FILM INCLUDING CELLULOSE AND METHOD OF MANUFACTURING THE SAME

(71) Applicant: WOORIKIDS+, INC., Yeonsu-gu (KR)

(72) Inventors: Hoisook KIM, Incheon (KR); Jungmin LEE, Suwon-si (KR); Hyojoo KIM, Incheon (KR); Milim KIM, Incheon (KR)

(73) Assignee: WOORIKIDS+, INC., Yeonsu-gu (KR)

(21) Appl. No.: 19/093,448

(22) Filed: Mar. 28, 2025

Related U.S. Application Data
(63) Continuation of application No. PCT/KR2025/002845, filed on Feb. 28, 2025.

(30) Foreign Application Priority Data
Feb. 29, 2024 (KR) 10-2024-0030163
Feb. 28, 2025 (KR) 10-2025-0026777

(51) Int. Cl. C08J 5/18; C08K 5/053

(52) U.S. Cl. CPC C08J 5/18; C08K 5/053; C08J 2301/02; C08J 2301/04; C08J 2305/00; C08J 2305/16

(57) ABSTRACT

Disclosed is a water-dissolvable film including cellulose according to various embodiments of the present invention for achieving the above-described tasks. The water-dissolvable film may be characterized by including pullulan, carboxymethyl cellulose, and cellulose.

### CROSS-REFERENCE TO RELATED PATENT APPLICATIONS

This application is a Continuation of International Application No. PCT/KR2025/002845, filed on Feb. 28, 2025, which claims the benefit of Korean Patent Application Nos. 10-2025-0026777, filed on Feb. 28, 2025, and 10-2024-0030163, filed on Feb. 29, 2024, in the Korean Intellectual Property Office, the disclosure of which is incorporated herein in its entirety by reference.

### TECHNICAL FIELD

The present invention relates to a water-dissolvable film, and more specifically, to an eco-friendly water-dissolvable film including cellulose, which can replace existing water-dissolvable films containing polyvinyl alcohol (PVA), and a method of manufacturing the same.

### BACKGROUND ART

Recently, the demand for water-dissolvable (water-soluble) films has been increasing in various industrial fields. Since water-dissolvable films have the characteristic of quickly dissolving when in contact with water under certain conditions and not remaining in the environment, they are widely utilized in various fields such as detergent packaging materials, pharmaceutical capsules, food packaging materials, and industrial coating materials. In particular, as existing plastic films have been pointed out as a main cause of environmental pollution, research on film materials that are biodegradable or water-dissolvable is actively progressing.

Currently, the most widely used water-dissolvable film material is polyvinyl alcohol (PVA). PVA has excellent water solubility and mechanical strength and has the characteristic of decomposing relatively quickly after dissolution, so it has been widely used in industry. For example, various products, such as laundry capsule films, pesticide packaging materials, medical water-dissolvable bags, and food packaging materials, are manufactured using PVA-based films.

However, recent research results have shown that PVA is water-soluble but has limitations in terms of biodegradability. In particular, a possibility that it will not be completely decomposed during the wastewater treatment process and will remain in the form of fine polymers and accumulate in the environment has been raised. According to reports from the U.S. Environmental Protection Agency (EPA) and some research institutes, about 75% of the PVA-based films are not completely decomposed after wastewater treatment and are released into the natural environment. For this reason, the European Union (EU), the United States, and other major countries are strengthening environmental regulations to limit the use of the PVA-based films.

Accordingly, it is essential to develop alternative materials that satisfies both water dissociation and mechanical strength without using PVA. However, existing research on alternative materials has several limitations. As a representative example, films based on natural polymers (e.g., cellulose, starch, chitosan, and the like) have excellent solubility in water, but have lower mechanical strength than PVA, making them brittle and prone to breaking. Conversely, some synthetic polymers have excellent mechanical strength, but have low water dissociation, which causes problems such as long dissolution times or residues.

In particular, manufacturing a water-dissolvable film without PVA is technically very difficult. PVA plays a role in optimizing the viscosity of the solution during the film formation process and helping to form a uniform thin film. That is, to manufacture a film without PVA, all of the following three key factors must be addressed: viscosity control, securing the mechanical strength of the film, and controlling water dissociation. In general, when the viscosity of a natural polymer-based film is too low, it becomes water-like and film formation is impossible, and when the viscosity is too high, a gelation phenomenon occurs and it is difficult to produce a uniform thin film. In addition, when the bonding strength between water-soluble polymers is weak, the film may tear easily or the mechanical strength may rapidly deteriorate.

Therefore, there is an urgent need to develop an alternative technology that can maintain physical properties similar to those of existing PVA-based films while not including PVA. In particular, in line with the recently strengthened environmental regulations, there is a need for new eco-friendly film materials that are completely biodegradable and do not cause microplastic emissions, and to achieve this, precise design in the film composition and manufacturing process is essential.

### DISCLOSURE

### Technical Problem

To solve the above-mentioned problems, the present invention is directed to overcoming the environmental limitations of polyvinyl alcohol (PVA)-based water-dissolvable films and providing an alternative film that can maintain excellent water dissociation and mechanical strength without PVA.

The problems of the present invention are not limited to the above-mentioned problem, and other problems not mentioned will be clearly understood by those skilled in the art from the following description.

### Technical Solution

A water-dissolvable film including cellulose according to various embodiments of the present invention for solving the above-described problems is disclosed. The water-dissolvable film may be characterized by including pullulan, carboxymethyl cellulose, and cellulose.

In an alternative embodiment, the water-dissolvable film may be characterized by including 2 parts by weight or more and 20 parts by weight or less of the carboxymethyl cellulose based on 100 parts by weight of the pullulan and including 2 parts by weight or more and 20 parts by weight or less of the cellulose based on 100 parts by weight of the pullulan.

In an alternative embodiment, the water-dissolvable film may be characterized by being formed using a mixed solution having a viscosity in a range of 1,500 centipoise (cps) or more and 6,500 centipoise or less.

In an alternative embodiment, the water-dissolvable film may be characterized by having a tensile strength in a range of 20 N or more and 50 N or less.

In an alternative embodiment, the water-dissolvable film may be characterized by forming an intermolecular hydrogen bonding network between the pullulan and the carboxymethyl cellulose.

In an alternative embodiment, the water-dissolvable film may be characterized by including main raw materials including the pullulan, the carboxymethyl cellulose, and the cellulose, and an auxiliary raw material for controlling the mechanical strength, flexibility, and dissolution characteristics of the film, wherein the auxiliary raw material is included in an amount of 100 parts by weight or less based on 100 parts by weight of the main raw materials.

In an alternative embodiment, the auxiliary raw material may include at least one of carrageenan, cyclodextrin, sorbitol, and glycerin.

In an alternative embodiment, the water-dissolvable film may be characterized by including an additional raw material including a surfactant, in which case the film has a final viscosity in a range of 15,000 centipoise or more and 30,000 centipoise or less.

According to another aspect of the present invention, a method of manufacturing a water-dissolvable film including cellulose is disclosed. The method may include preparing a mixed solution including pullulan, carboxymethyl cellulose, and cellulose, applying the mixed solution onto a flat plate, and drying the applied mixed solution to form a water-dissolvable film.

### Advantageous Effects

According to various embodiments of the present invention, a water-dissolvable film that can implement a viscosity and tensile strength similar to those of the existing PVA-based film without PVA can be provided. This has the effect of maintaining the mechanical strength and processability of the film while addressing the environmental concerns of the existing PVA-based films.

In addition, the film of the present invention is completely biodegradable and has eco-friendly characteristics that do not cause environmental pollution such as microplastics even during wastewater treatment. Accordingly, it can respond to increasingly strengthened global environmental regulations and can be utilized as a sustainable alternative material in various industrial fields.

In addition, the physical properties of the film can be further improved by combining auxiliary raw materials as needed. For example, the elasticity and durability of the film can be improved by adding specific softeners and crosslinking agents, and the processability during film formation can be improved by introducing a viscosity modifier. By this composition control, the physical properties of the film can be optimized for the intended purpose.

Effects of the present invention are not limited to the effects mentioned above, and other effects not mentioned will be clearly understood by those skilled in the art from the description below.

### DESCRIPTION OF DRAWINGS

Various aspects are now described with reference to the drawings, wherein like reference numerals are used to generally refer to similar components. In the following embodiments, for illustrative purposes, a number of specific details are set forth to provide a comprehensive understanding of one or more aspects. However, it will be clear that such aspects may be implemented without these specific details.

FIG. 1 illustrates an exemplary flow chart of a method of manufacturing a water-dissolvable film including cellulose according to one embodiment of the present invention.

FIG. 2 is an exemplary diagram illustrating pullulan according to one embodiment of the present invention.

FIG. 3 is an exemplary diagram illustrating carboxymethyl cellulose according to one embodiment of the present invention.

FIG. 4 is an exemplary diagram illustrating cellulose according to one embodiment of the present invention.

FIG. 5 is an exemplary diagram illustrating a process of manufacturing a water-dissolvable film including cellulose according to one embodiment of the present invention.

FIG. 6 is an exemplary diagram illustrating a water-dissolvable film including cellulose according to one embodiment of the present invention.

FIGS. 7 to 9 are drawings showing films produced as experimental results according to one embodiment of the present invention.

FIG. 10 is an exemplary diagram showing a tensile strength (tensile stress)-strain curve of a water-dissolvable film including cellulose according to one embodiment of the present invention.

FIG. 11 is a drawing showing a film manufactured by adding a surfactant to a water-dissolvable film composition according to one embodiment of the present invention.

### BEST MODES OF THE INVENTION

Various embodiments and/or aspects are now disclosed with reference to the drawings. In the following description, for purposes of explanation, numerous specific details are set forth to provide a general understanding of one or more aspects. However, it will also be apparent to those skilled in the art that these aspects may be practiced without these specific details. The following description and the attached drawings describe certain exemplary aspects of one or more of the aspects in detail. However, these aspects are illustrative and some of the various methods in the principles of various aspects may be utilized, and the descriptions are intended to include all such aspects and their equivalents. Specifically, the terms “embodiment,” “example,” “aspect,” and the like as used herein may not be construed to imply that any aspect or design described is better or advantageous over other aspects or designs.

Hereinafter, regardless of the drawing symbol, identical or similar components are given the same reference number and redundant descriptions thereof are omitted. In addition, when describing the embodiments disclosed in this specification, when it is determined that a detailed description of a related known technology may obscure the subject matter of the embodiments disclosed in this specification, the detailed description is omitted. In addition, the attached drawings are only provided to facilitate easy understanding of the embodiments disclosed in this specification, and the technical ideas disclosed in this specification are not limited by the attached drawings.

Objects and advantages of the present invention, and technical configurations for achieving them, will become apparent with reference to the embodiments described in detail below in conjunction with the accompanying drawings. In the description of the present invention, when it is determined that a specific description of a known function or configuration may unnecessarily obscure the gist of the present invention, the detailed description thereof will be omitted. Also, the following terms are terms defined in consideration of functions in the present invention, which may vary according to the user, the intent or practice of the operator, etc.

However, the present invention is not limited to the embodiments disclosed below, but may be embodied in various different forms. The embodiments are merely provided to complete the present invention and to fully convey the scope of the invention to those skilled in the art, and the invention is only defined by the scope of the claims. Therefore, the definition should be made on the basis of the contents throughout this specification.

Water-dissolvable (water-soluble) films are widely utilized in various industrial fields, and are attracting attention, in particular, in the fields of detergent packaging materials, food packaging materials, pharmaceutical capsules, and industrial coatings. Polyvinyl alcohol (PVA) is used as the main raw material for existing water-dissolvable films, and PVA has excellent film forming ability and mechanical strength, providing a stable manufacturing process and excellent performance.

However, despite its solubility in water, PVA is reported to be difficult to completely biodegrade in the environment. In particular, as the possibility arises that some of the undecomposed PVA may be released into the environment during the wastewater treatment process, the need for eco-friendly materials that can replace PVA is growing. Accordingly, natural polymer-based films are being considered as alternatives, but existing natural polymer films have problems such as low mechanical strength or poor processability, making practical application difficult.

The present invention is intended to provide a water-dissolvable film that can maintain a viscosity and tensile strength similar to those of the existing PVA-based films without including PVA, and for this purpose, a method of manufacturing a film based on a specific composition is proposed.

The water-dissolvable film of the present invention is an eco-friendly film configured to implement a mechanical strength and viscosity similar to those of the existing PVA-based film without including PVA. The water-dissolvable film of the present invention is based on a composition including cellulose, and the composition ratio and manufacturing process are optimized to facilitate film formation while maintaining a certain viscosity range. In addition, the physical properties of the film may be adjusted by combining the auxiliary raw materials, thereby controlling the mechanical strength, flexibility, and dissolution rate of the water-dissolvable film.

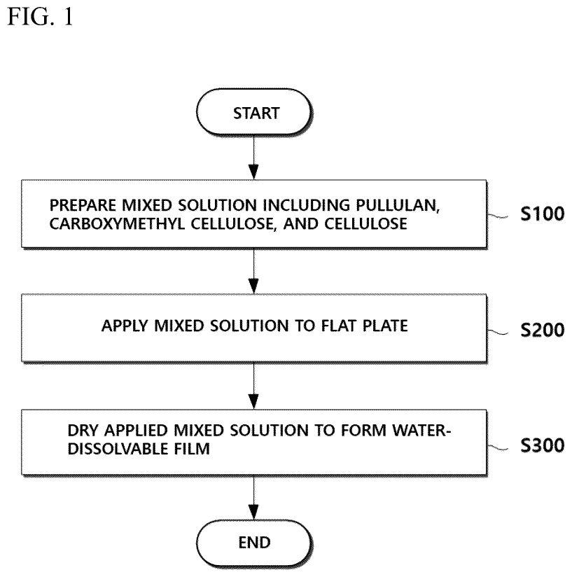

A specific description of the water-dissolvable film of the present invention and a method of manufacturing the same will be described below with reference to FIGS. 1 to 6.

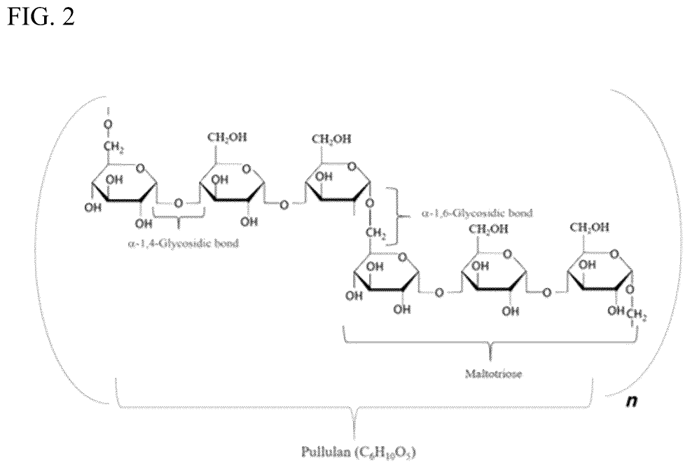

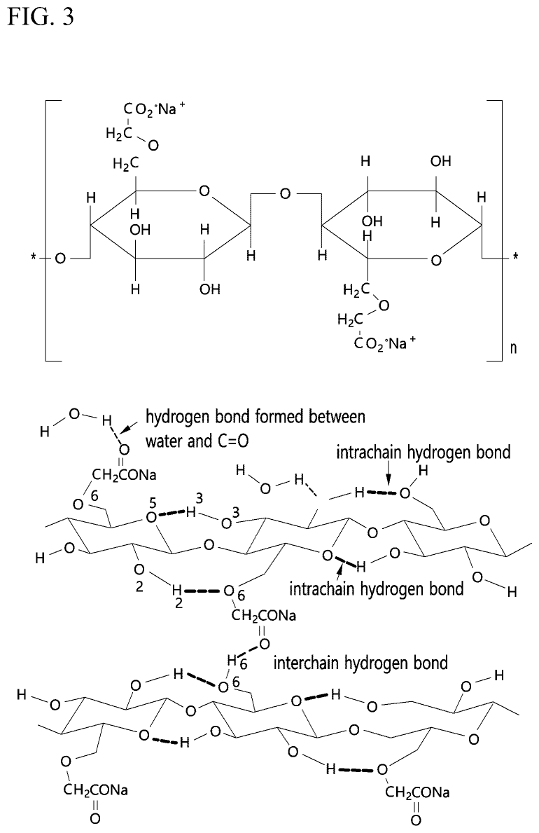

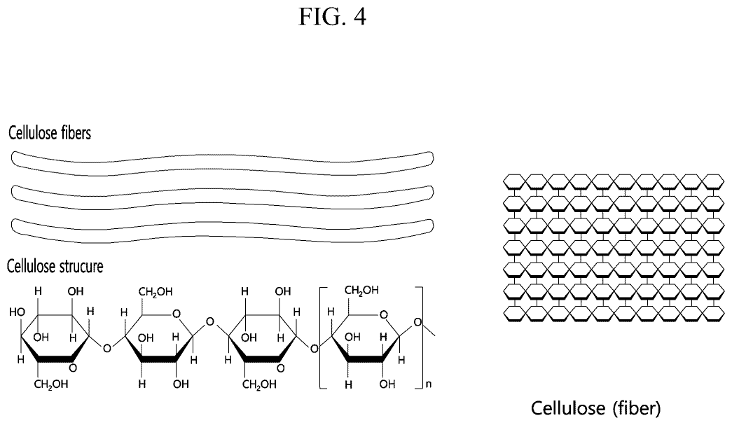

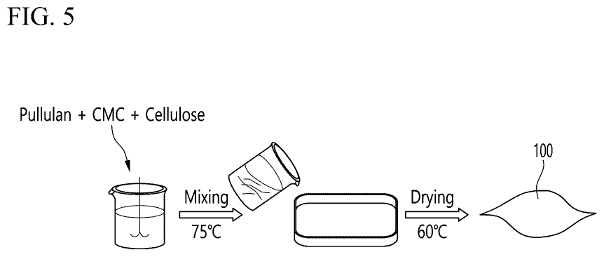

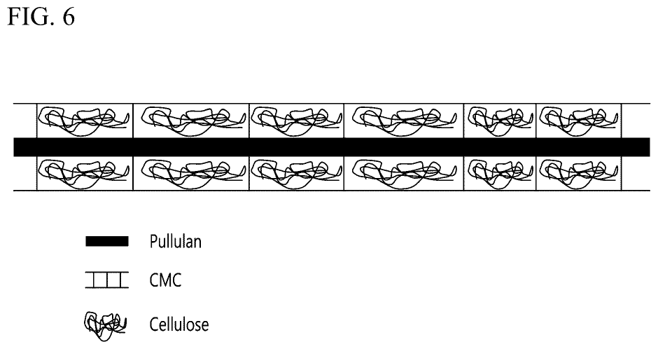

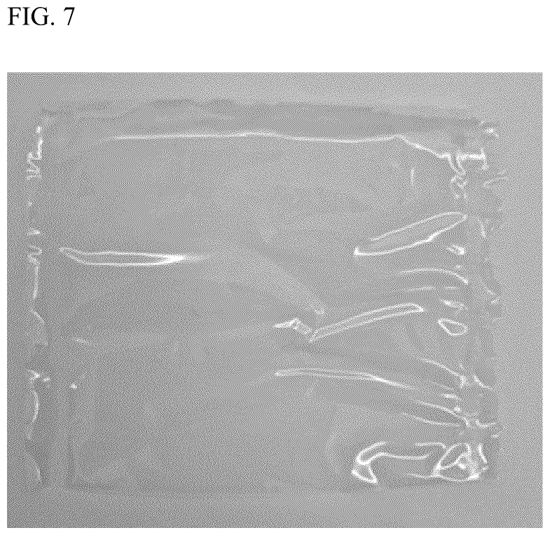

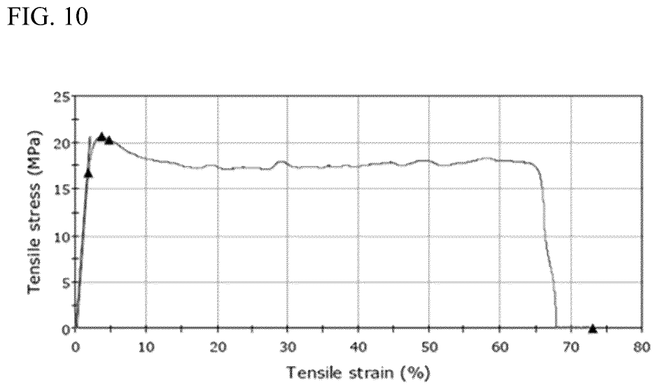

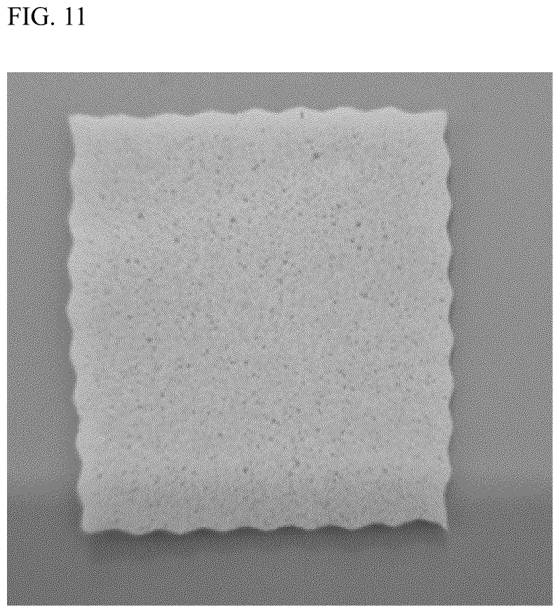

FIG. 1 illustrates an exemplary flow chart of a method of manufacturing a water-dissolvable film including cellulose according to one embodiment of the present invention. FIG. 2 is an exemplary diagram illustrating pullulan according to one embodiment of the present invention. FIG. 3 is an exemplary diagram illustrating carboxymethyl cellulose according to one embodiment of the present invention. FIG. 4 is an exemplary diagram illustrating cellulose according to one embodiment of the present invention. FIG. 5 is an exemplary diagram illustrating a process of manufacturing a water-dissolvable film including cellulose according to one embodiment of the present invention. FIG. 6 is an exemplary diagram illustrating a water-dissolvable film including cellulose according to one embodiment of the present invention. FIGS. 7 to 9 are drawings showing films produced as experimental results according to one embodiment of the present invention. FIG. 10 is an exemplary diagram showing a tensile strength (tensile stress)-strain curve of a water-dissolvable film including cellulose according to one embodiment of the present invention. FIG. 11 is a drawing showing a film manufactured by adding a surfactant to a water-dissolvable film composition according to one embodiment of the present invention.

Referring to FIG. 1, the method of manufacturing a water-dissolvable film including cellulose may include a step of preparing a mixed solution including pullulan, carboxymethyl cellulose (CMC), and cellulose (S100).

In the present invention, the preparation of the mixed solution is a key process for film formation, and it is important to ensure that each component is homogeneously dispersed and an optimal viscosity range is formed.

The step of preparing a mixed solution is characterized by dissolving each component in an appropriate ratio and mixing it homogeneously to maintain a viscosity suitable for film formation. For this purpose, a process may be included in which each component is sequentially added to a solvent heated to 50 to 80° C. and stirred at a constant speed to form a uniform composition.

In one embodiment, a certain amount of distilled water is prepared in a reaction vessel and then heated to a temperature of 50 to 80° C. so that the mixed components may be smoothly dissolved. Afterwards, after adding pullulan, stirring may be performed at a speed of 1,500 rpm to form a homogeneous solution.

Referring to FIG. 2, the pullulan used in method of manufacturing a water-dissolvable film of the present invention is a natural polysaccharide, which is composed of continuous bonding of triglucoside units having α-(1→6) and α-(1→4) glycosidic bonds. Due to these structural features, it is evaluated as a material with excellent water solubility, film forming ability, and excellent compatibility with other biodegradable polymers.

Pullulan may form a colorless transparent film and has eco-friendly properties as a natural component. In addition, it has a high oxygen barrier property, which plays a role in improving moisture retention and preservation within the film. Due to these characteristics, it may be utilized in various fields such as functional packaging materials, food protective films, and medical films.

Referring to the structural formula of FIG. 2, pullulan has a form in which triglucoside units are linked through α-(1→4)-glycosidic bonds and α-(1→6)-glycosidic bonds, which plays a role in maximizing water solubility and film forming ability. In addition, due to these structural features, the compatibility with other biodegradable polymers (e.g., carboxymethyl cellulose and cellulose) is excellent, and through this, the strength and dissolution characteristics of the water-dissolvable film of the present invention may be controlled.

That is, pullulan is a natural polysaccharide with excellent water solubility and film forming ability, and plays a role in controlling viscosity and forming a homogeneous structure of a water-dissolvable film 100.

In addition, in the embodiment, a certain amount of CMC may be further added to a solution in which pullulan is homogeneously dissolved, and stirred at a speed of 1,000 to 2,500 rpm to form a homogeneous solution.

In the process of manufacturing the water-dissolvable film of the present invention, CMC serves to control the physical properties of the film by being mixed with pullulan and cellulose. CMC combines with pullulan to maintain a constant viscosity while reinforcing the strength of the film, making film formation easy. CMC is a hydrophilic polymer, functions to control the viscosity of the solution, and provides structural stability during the film formation process.

Referring to FIG. 3, CMC is generally present in the form of a sodium salt (CMC-Na) and has high compatibility with water. In addition, when CMC is mixed with pullulan, it may form hydrogen bonds to form a homogeneous network structure within the film, thereby improving the mechanical strength of the film.

More specifically, hydrogen bonding within CMC may be largely divided into two types.

Intrachain hydrogen bonds are bonds formed between a hydroxyl group (—OH) and a carboxymethyl group (—CH₂COO) within a single CMC molecule, which contribute to maintaining the structural stability of the polymer chain and serve to prevent excessive swelling of the film when absorbing moisture.

In addition, intermolecular hydrogen bonds are bonds formed between CMC molecules, which serve to improve the mechanical strength of the film by forming a multi-polymer network. In particular, this bonding structure increases the durability of the film and has a characteristic that differentiates it from the reinforcing method that utilizes inorganic ions such as phosphorus (P), iron (Fe), magnesium (Mg), and iodine (I) that were utilized to reinforce mechanical strength in the existing PVA-based film.

According to an embodiment, CMC may form hydrogen bonds with external moisture, thereby maintaining constant mechanical strength within a specific moisture content while maintaining the flexibility of the film.

According to an embodiment of the present invention, when CMC and pullulan are mixed, the polysaccharide structure of pullulan and the carboxymethyl group (—CH₂COO—) of CMC may interact to form a stronger hydrogen bond. Hydrogen bonds formed between pullulan and CMC may contribute to maintaining a homogeneous network structure within the film. CMC may have low mechanical strength when used alone, but when it is used together with pullulan and cellulose, it can effectively improve the tensile strength of the film by forming multiple hydrogen bonds.

In addition, unlike an inorganic ion-based reinforcement method utilized in the existing PVA-based film, the present invention utilizes hydrogen bonds based on natural polysaccharides to secure further eco-friendliness and excellent mechanical properties. Accordingly, the water-dissolvable film of the present invention is optimized to be able to dissolve within a certain period of time when in contact with water while maintaining strength based on the interaction between CMC and pullulan.

According to an embodiment of the present invention, cellulose may improve the physical properties of the film and maintain a homogeneous network structure by being mixed with pullulan and CMC.

In one embodiment, pullulan and carboxymethyl cellulose may be characterized by forming an intermolecular hydrogen bonding network in a mixed solution, thereby inducing uniform structural formation of a water-dissolvable film and improved mechanical strength.

According to an embodiment, cellulose may include crystalline cellulose, which may reinforce the mechanical strength of the film and maintain structural stability.

For example, the crystalline cellulose may be a form of cellulose in which an amorphous region is removed from natural cellulose and high crystallinity is maintained. The crystalline cellulose has high mechanical strength through strong hydrogen bonds between cellulose chains and may be utilized as a reinforcing agent for various biodegradable films and eco-friendly materials.

That is, the crystalline cellulose has high crystallinity and may play a role in increasing strength and durability within the film. In addition, it may form a uniform network through interactions with fluorine and CMC, and contribute to maintaining the tensile strength and durability of the film.

More specifically, cellulose is a natural polymer, provides excellent mechanical strength, and acts as a component that maintains the structural stability of the film. Cellulose is basically a water-soluble polymer polymer, but due to its high crystallinity and strong hydrogen bonding network, it is not readily soluble in water alone. These features are an important factor in increasing mechanical strength by maximizing the bonding strength between polymer chains within the film. The alignment of cellulose fibers is maintained by multiple hydrogen bonds formed between the polymer chains, which is the reason why it exhibits the highest strength among cellulose-based structures.

Referring to FIG. 4, cellulose is a linear polymer composed of repeating glucose (β-D-glucopyranose) units of polysaccharides, which forms a strong hydrogen bonding network and exhibits high structural stability. In particular, cellulose has excellent moisture absorption capacity, so it can provide various functionalities depending on the composition of the film, and when a crosslinked structure is formed, it may secure flexibility while maintaining the strength of the film.

Referring to the molecular structure of FIG. 4, cellulose is a linear polymer composed of continuous β(1→4) glycosidic bonds and includes a large amount of hydroxyl groups (—OH), which may form strong interactions with pullulan and CMC.

In an embodiment, cellulose has the property of branching and linking in a manner similar to a paper fiber structure, and this structural feature may play an important role in maintaining high tensile strength within the film.

In addition, cellulose may play an important role in controlling the mechanical strength of the sheet when applied together with other filler components. For example, when fillers such as mannitol and xylitol are added, the mechanical strength of the film can be supplemented, but when a certain ratio is exceeded, a problem of increased stickiness may occur. Accordingly, in embodiments of the present invention, the ratio of cellulose may be optimized to adjust additional physical properties while maintaining the strength of the film.

As described above, the mixed solution of the present invention includes pullulan, CMC, and cellulose as main components, and may be configured to form a homogeneous network structure through interactions between these components. More specifically, pullulan is a natural polysaccharide with excellent film forming ability and plays a role in providing mechanical stability to the film, and CMC has excellent water solubility and can provide the function of optimizing a film formation process by controlling the viscosity of the solution. In addition, cellulose forms a strong hydrogen bonding network to maximize the mechanical strength of the film and plays a role in controlling the physical properties of the film through interactions with pullulan and CMC.

According to one embodiment, the mixed solution may be characterized by including 30 parts by weight or less of carboxymethyl cellulose and 30 parts by weight or less of cellulose, based on 100 parts by weight of pullulan. That is, in the mixed solution, CMC and cellulose may be included in an amount of 30 wt % or less relative to 100 wt % of pullulan. This composition ratio may contribute to optimizing the viscosity of the mixed solution during the film formation process and maintaining the homogeneous network structure within the film.

According to various embodiments, the mixed solution of the present invention may be composed of a combination of main raw materials including pullulan, carboxymethyl cellulose, and cellulose, and an auxiliary raw material for controlling the mechanical strength, flexibility, and dissolution characteristics of the film. The water-dissolvable film of the present invention may be configured to secure the structural stability of the film through interactions between main raw materials, and to more precisely control the physical properties of the film by adding the auxiliary raw material.

According to an embodiment, the auxiliary raw material included in the mixed solution may include at least one of carrageenan, cyclodextrin, sorbitol, and glycerin. Carrageenan is a natural polysaccharide and can play a role in increasing the viscosity within the film and controlling the dissolution rate, and cyclodextrin plays a role in reinforcing the hydrogen bonding network to improve the strength and durability of the film. In addition, sorbitol and glycerin can contribute to improving the flexibility of the film and maintaining appropriate elasticity even after the film is dried.

According to an embodiment of the present invention, the auxiliary raw material may be included in an amount of about 100 parts by weight (preferably 100 parts by weight or less) based on 100 parts by weight of the main raw materials, and may be configured to maintain the mechanical strength and dissolution characteristics of the film in a balanced manner by being blended in an appropriate ratio in the mixed solution. In addition, the content of the auxiliary raw material may act as an important factor in controlling the final properties of the film, and an optimal mixing ratio may be derived to secure a certain degree of water dissociation while maintaining the durability of the film.

As a specific example, carrageenan may be included in an amount ranging from 0.1 to 2.0 parts by weight relative to the sum of the main and auxiliary raw materials, and may serve to increase the viscosity of the film and control the dissolution rate. When the content of carrageenan is too high, the viscosity of the film may increase excessively, which may result in poor uniformity during the application process, and conversely, when the content is too low, the structural stability of the film may decrease.

In addition, in one embodiment, cyclodextrin is a cyclic oligosaccharide having a hydrophilic exterior and a hydrophobic interior and serves to reinforce the hydrogen bonding network within the film of the present invention, thereby improving the strength and durability of the film. For example, when the content of cyclodextrin is included in a range of 0.5 to 5.0 parts by weight relative to the sum of the main and auxiliary raw materials, the physical properties of the film are balanced, and when it is too much or too little, the tensile strength and flexibility of the film may be affected.

In particular, cyclodextrin has the characteristic of forming hydrogen bonds with sodium ions and plays an important role in strengthening the polymer network within the film. Specifically, hydrogen bonds are formed between the hydroxyl group (—OH) of CMC and cellulose and the hydroxyl group (—OH) of cyclodextrin, and at the same time, the sodium ions (Na) of CMC bind to cyclodextrin to reinforce the network structure.

In this process, the porous structure of cyclodextrin promotes the formation of hydrogen bonds within the film, and the carboxymethyl group (—COO⁻) and sodium ion (Na+) of CMC combine with the hydroxyl group (—OH) of cyclodextrin to form a stable polymer network. This binding method may further increase the structural stability of the film compared to when CMC-Na⁺ is present alone, and effectively improve the physical strength compared to the existing PVA-based film.

In addition, since cyclodextrin has a structure that may form numerous hydrogen bonds, it can also interact with pullulan and CMC to form a denser network within the film. When a cyclodextrin content exceeds an appropriate range, excessive hydrogen bonding may be formed, which may excessively increase the tensile strength of the film, thereby reducing flexibility and making the film brittle. Conversely, when the cyclodextrin content is too low, the network formation within the film may be incomplete, resulting in insufficient structural stability.

In an embodiment of the present invention, cyclodextrin may be included in a range of 0.5 to 5.0 parts by weight to optimize the hydrogen bonding network of the film and maintain a balance between strength and flexibility. In particular, the multiple hydrogen bonding network formed by cyclodextrin combining with CMC-Na⁺ and cellulose may contribute to maintaining uniform physical properties of the film and controlling interactions with water.

In addition, in the embodiment, sorbitol and glycerin may be included in an amount ranging from 0.5 to 10.0 parts by weight, respectively, and may contribute to improving the flexibility of the film and maintaining appropriate elasticity even after drying. When the content of sorbitol and glycerin is excessive, the surface of the film may become sticky, and conversely, when the content is too low, the film may become excessively hardened and become brittle.

In an embodiment of the present invention, by blending the above-mentioned auxiliary raw materials in a ratio of about 100 parts by weight (preferably 100 parts by weight or less) based on 100 parts by weight of the main raw materials (pullulan, CMC, cellulose), the mechanical strength, flexibility, and water dissociation property of the film may be balanced.

In particular, the mixed solution of the present invention may be configured to provide excellent physical properties by utilizing eco-friendly components while resolving difficulties in controlling mechanical strength and dissolution rate that appear in an existing PVA-based film.

In this embodiment, pullulan acts as a component that forms the main skeleton of the film and can provide excellent film forming ability. Carboxymethyl cellulose can control the viscosity of the mixed solution and enable uniform application during the film forming process, and cellulose can provide high mechanical strength to improve the durability of the film.

In addition, the mixed solution of the present invention may be stirred at a constant temperature (e.g., 50 to 80° C.) to form a homogeneous solution, and during the process, as hydrogen bonds are formed between pullulan and CMC, the network structure within the film may be stably maintained.

In particular, by controlling the viscosity of the mixed solution to a range of 1,500 centipoise (cps) or more and 6,500 centipoise or less, the tensile strength of the film may be optimized and the mechanical strength may be uniformly maintained. In a more specific embodiment, the viscosity of the mixed solution is preferably 2,200 centipoise or more and 3,000 centipoise or less. This is in a similar range to the mixed solution viscosity (e.g., 2,710 cps) of the PVA-based film, and is effective in maintaining uniform formation and appropriate mechanical strength of the film. That is, by controlling the viscosity of the mixed solution to a level similar to that of the PVA-based film (in a range of about 2,200 cps to 3,000 cps), the flexibility and processability of the film may be maintained while securing mechanical strength.

According to an embodiment, when the viscosity is too low, the fluidity of the mixed solution increases, resulting in insufficient network formation within the film, which may deteriorate the structural stability of the film. Conversely, when the viscosity is too high, the mixed solution may gelate, causing agglomeration within the film, making it difficult to form a uniform network, and resulting in uneven tensile strength.

In an embodiment of the present invention, by controlling the viscosity of the mixed solution to an appropriate range, the hydrogen bonding between pullulan, CMC, and cellulose may be optimized during the film forming process, and accordingly, the resulting film may have a tensile strength in a range of 20 N or more and 50 N or less.

In a more specific embodiment, in order to secure a mechanical strength similar to that of the PVA-based film while maintaining solubility and environmental friendliness, it is preferable that the tensile strength is in a range of 35 N or more and 45 N or less.

This is an appropriate range that allows the water-dissolvable film of the present invention to secure structural stability while maintaining mechanical strength compared to the PVA-based film (e.g., 44.80 N). When the tensile strength is less than 35 N, the durability of the film may be reduced, and when it exceeds 45 N, the flexibility of the film may be reduced. Therefore, through the composition of the present invention, an optimal tensile strength range of 35 N to 45 N may be secured, thereby providing a film having a balance between mechanical strength and water dissociation.

According to various embodiments, the mixed solution of the present invention may be characterized by including an additional raw material including a surfactant, wherein when the additional raw material is included, the final viscosity is in a range of 15,000 centipoise or more and 30,000 centipoise or less.

In an embodiment, the additional raw material may include cosmetic raw materials, functional additives, active components, moisturizers, biodegradation accelerators, physiologically active components, and other raw materials with industrial applications, as components for controlling the physical properties of the film.

For example, when the surfactant is included as an additional raw material, the film may be utilized as a cosmetic sheet such as a cleansing sheet. In addition, when a sodium component such as sodium carbonate is included, it may be manufactured in a sheet form that has the property of dissolving in water, so it may be utilized for various purposes. These compositions allow the functionality of the film to be adjusted to suit specific industrial needs. Specifically, in an embodiment of the present invention, the additional raw material may include one or more of a moisturizer (e.g., hyaluronic acid, glycerin, and propylene glycol), an antioxidant (e.g., a vitamin C derivative and tocopherol), a physiologically active component (e.g., a peptide and flavonoid), a surfactant (e.g., lecithin and sodium lauryl sulfate), a functional additive (e.g., zinc oxide and titanium dioxide), and other components having industrial applications.

In particular, when the additional raw material is included, the mechanical strength and flexibility of the film may be changed while the final viscosity of the film may be controlled, and the additional raw material may be configured to enable control of the dissolution rate and release of specific components.

For example, when the moisturizer is included to control moisture content, the flexibility of the film may be increased and thus the tensile strength may be controlled, and when the surfactant is included, the dissolution rate of the film may be adjusted.

In addition, when the final viscosity is adjusted to 15,000 cps or more, the network within the film may be more firmly formed, and the strength and durability of the film may be improved. However, when the viscosity exceeds 30,000 cps, the viscoelasticity of the solution may increase excessively, which may deteriorate the processability in the film forming process, and conversely, when the viscosity is less than 15,000 cps, the mechanical strength of the film may deteriorate.

That is, when the additional raw material is included in the mixed solution of the present invention, the mechanical strength, flexibility, and dissolution characteristics of the film may be controlled, and optimal physical properties may be secured by maintaining the final viscosity at 15,000 cps or more and 30,000 cps or less. The functionality of the film may vary depending on the composition and proportion of additional raw materials, and the inclusion of specific components may adjust the water dissociation rate and release behavior of the film.

In addition, in an embodiment, the method of manufacturing a water-dissolvable film including cellulose may include a step of applying the mixed solution onto a flat plate (S200). The application process may include a step of forming a uniform thickness of the film and optimizing tensile strength and water dissociation.

Specifically, the mixed solution may be quantitatively applied onto a flat plate (e.g., a glass plate, a silicone mold, a metal plate, and the like) so as to uniformly maintain film thickness and mechanical strength, and the application process may be performed while maintaining a strategically set viscosity (1,500 cps to 6,500 cps or 15,000 cps to 30,000 cps when including additional raw materials).

According to an embodiment, an application method may include at least one of a casting method, a roll coating method, a doctor blade method, and a slot die coating method. The casting method is mainly suitable for manufacturing uniform films in laboratory environment, while the roll coating or slot die coating method may be applied for mass production.

After the application process is completed, the mixed solution may be dried for a certain period of time in a range of 40 to 60° C. to form a film, during which the hydrogen bonding network between the pullulan, CMC, cellulose, and other auxiliary raw materials may be strengthened. In addition, the coating thickness may affect the final mechanical properties and water dissociation of the film, and according to an embodiment, a thickness of the film may be set in the range of 20 μm to 200 μm, but is not limited thereto.

In an embodiment of the present invention, the viscosity of the mixed solution may be controlled to an optimal range during the application process so that a constant tensile strength is maintained even after the film is dried.

In addition, in an embodiment, the method of manufacturing a water-dissolvable film including cellulose may include a step of drying the applied mixed solution to form a water-dissolvable film (S300).

According to an embodiment, the water-dissolvable film 100 of the present invention has a tensile strength in a range of 20 N or more and 50 N or less, which may be implemented in an eco-friendly manner while maintaining a mechanical strength similar to that of the existing PVA-based film.

The PVA-based film has been widely used in existing industries, but its environmental persistence has become a problem. The water-dissolvable film 100 of the present invention is configured to solve these problems while achieving a tensile strength similar to that of an existing PVA film.

More specifically, the applied mixed solution may be dried at a temperature ranging from 40 to 60° C. for a certain period of time, during which the hydrogen bonding network between the pullulan, carboxymethyl cellulose, and cellulose may be strengthened. In addition, the drying rate and temperature may directly affect the mechanical properties of the film, and in an embodiment of the present invention, a drying time may be set in a range of 2 hours or more and 24 hours or less.

The dried film is configured to maintain water dissociation while securing tensile strength and can provide equivalent or improved physical properties compared to those of the existing PVA-based film. In particular, unlike PVA films, the water-dissolvable film of the present invention may secure mechanical strength through hydrogen bonding between natural polysaccharides without a chemical crosslinking agent, and may be naturally decomposed after use.

In summary, as shown in FIG. 5, the method of manufacturing a water-dissolvable film of the present invention may include a process of mixing a mixed solution including pullulan, carboxymethyl cellulose, and cellulose at about 75° C. and then drying the resulting mixture at about 60° C. to form a film. In the manufacturing process, a homogeneous solution is formed at the mixing step, and the mechanical strength and water dissociation of the film may be controlled at the application and drying steps.

The water-dissolvable film manufactured through the above-described process may have a multilayer structure in which pullulan forms the main skeleton of the film, CMC constitutes the internal network, and cellulose fibers provide structural support as shown in FIG. 6. This structural arrangement may serve to increase the tensile strength of the film through hydrogen bonding and to control water dissociation under certain temperature and humidity conditions.

The water-dissolvable film of the present invention may be formed using eco-friendly raw materials while having similar physical properties to the PVA-based film, and the mechanical strength, flexibility, and dissolution characteristics of the film may be controlled. Further description on this will be provided in detail in the experimental contents below.

### Experimental Method and Result Analysis (First Experiment)

In order to evaluate the mechanical properties of the water-dissolvable film of the present invention, tensile strength and viscosity measurement experiments were performed. A polyvinyl alcohol (PVA)-based film was set as the comparative group for the experiment, and the physical properties of the PVA-based film were analyzed in comparison with those of the pullulan, carboxymethyl cellulose, and cellulose-based water-dissolvable films of the present invention.

### 1. Experimental Equipment and Conditions

In order to evaluate the mechanical properties of the water-dissolvable film of the present invention, tensile strength and viscosity measurement experiments were performed. To measure the tensile strength, a universal testing machine (Instron Korea 5569 model) was used, and a 1 kN load cell and air pneumatic grips were applied to fix the specimen. A test speed was set to 3,000 mm/min, and the test piece was manufactured to have a width of 10 mm, a thickness of 0.210 mm, and a length of 30 mm to perform the experiment.

Viscosity measurements were performed using a CAS model CL-R2 viscometer, and the experimental conditions were a rotational speed of 20 rpm and a measurement temperature of 25° C. Through this, the viscosity properties of the mixed solution of the present invention were evaluated and compared to the PVA-based film, and at the same time, the differences in viscosity were analyzed from various compositions in which pullulan, CMC, cellulose, and other cellulose derivatives (such as HPMC) were combined. Through these comparisons, the effects on film formability, mechanical strength, and water dissociation were comprehensively evaluated.

### 2. Preparation of Mixed Solution and Film Formation

In order to evaluate the mechanical strength, viscosity, and water dissociation of the water-dissolvable film of the present invention, mixed solutions of various compositions were prepared, applied onto a flat plate, dried to form films, and then the physical properties were measured. For comparative experiments, the existing PVA-based film (Comparative Example 1), a film of the present invention (Experimental Example 1), and films of various compositions (Experimental Example 2) were manufactured and their physical properties were comparatively analyzed.

### (1) Comparative Example 1: PVA-Based Film Manufacturing and Physical Property Evaluation

An existing PVA-based film was manufactured and set as a standard for comparison with the film of the present invention. In general, PVA has excellent water solubility and film forming ability, but as a chemically synthesized polymer, it has the problem of low biodegradability and environmental sustainability. Therefore, based on Comparative Example 1, the film of the present invention was evaluated for its potential as an eco-friendly alternative material while having physical properties similar to those of a PVA film.
Composition: 12 wt % of polyvinyl alcohol (PVA) and 88 wt % of distilled water
Manufacturing method: After dissolving PVA in distilled water, a mixed solution was applied onto a flat plate and dried to form a film.

### (2) Experimental Example 1: Manufacturing and Physical Property Evaluation of Water-Dissolvable Film of Present Invention

The water-dissolvable film of the present invention was manufactured by combining pullulan, carboxymethyl cellulose, and cellulose in a specific ratio. In the present invention, pullulan plays a role in forming the main skeleton of the film, CMC contributes to controlling viscosity and increasing flexibility, and cellulose plays a role in reinforcing the mechanical strength of the film.
Composition: CMC and cellulose are each included in an amount ranging from 2 to 20 parts by weight based on 100 parts by weight of pullulan. Specifically, the film includes 80 wt % of pullulan, 10 wt % of CMC, and 10 wt % of cellulose, based on 100 wt % of the total composition
Manufacturing method: A mixed solution was prepared by mixing pullulan, CMC, and cellulose in a certain ratio, then applied onto a flat plate and dried to form a film.

The water-dissolvable film of the present invention formed through the above process is as shown in FIG. 7.

### (3) Experimental Example 2: Experiment Comparing Various Compositions

Films were manufactured at various ratios to compare with the optimal composition of the present invention. In Experimental Example 2, several combinations were set to confirm the change in film properties according to changes in individual component composition and additives.

In Experimental Example 2, hydroxypropyl methylcellulose (HPMC) was additionally utilized as another cellulose to increase the structural stability of the film and control the viscosity of the solution to enable uniform film formation. HPMC is a widely used component in a hydrogel sheet, has the property of gelating the entire solution, and may play a role in increasing stability in a cold air process. Through this, it was intended to strengthen the mechanical strength of the film and secure physical properties under specific process conditions.

### 1) Composition Including Only Pullulan and CMC (1-1)

Composition: The composition includes only pullulan and CMC, excluding cellulose. The composition was prepared by mixing pullulan and CMC in a ratio of 8:1.

Purpose: Confirmation of effects of viscosity control and hydrogen bonding of CMC on the strength and water dissociation of pullulan-based films.

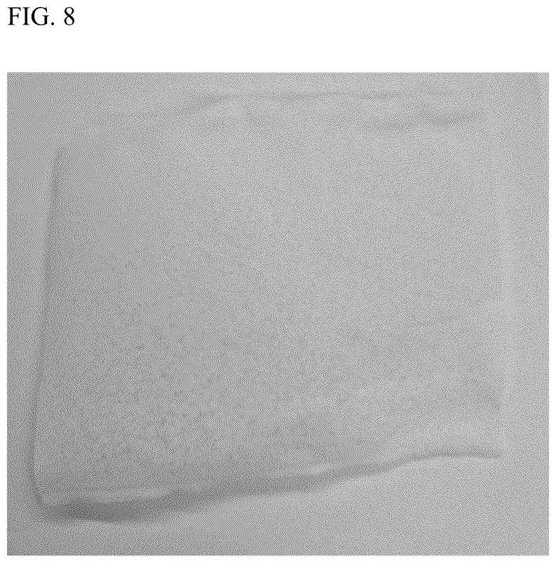

The film manufactured by utilizing the composition prepared by this composition is as shown in FIG. 8.

### 2) Composition Including Only Pullulan and Cellulose (1-2)

Composition: The composition includes pullulan and cellulose, excluding CMC. The composition was prepared by mixing pullulan and cellulose in a ratio of 8:1.

Purpose: Evaluation of changes in the mechanical strength and physical properties of the film when cellulose is added alone

### 3) Composition Including Pullulan and Another Cellulose (HPMC) (1-3)

Composition: The composition includes pullulan and HPMC instead of basic cellulose. The composition was prepared by mixing pullulan and HPMC in a ratio of 8:1.

Purpose: Analysis of the effect of HPMC on water dissociation and mechanical strength.

### 4) Composition Including Pullulan, CMC, and Another Cellulose (HPMC) (1-4)

Composition: The composition includes pullulan, CMC, and HPMC. The composition includes pullulan, CMC, and HPMC in a ratio of 8:1:1.

Purpose: Confirmation of effect of bonding between CMC and HPMC on physical properties of film.

### 5) Composition Including Pullulan, CMC, Cellulose, and Another Cellulose (HPMC) (1-5)

Composition: The composition includes pullulan, CMC, cellulose, and HPMC. The composition includes pullulan, CMC, cellulose, and HPMC in a ratio of 8:1:1:1.

Purpose: Analysis of how the mechanical strength and water dissociation of a film change when four components are combined.

### 6) Composition Including Only CMC and Cellulose (1-6)

Composition: The composition consists only of CMC and cellulose, excluding pullulan. The composition was prepared by mixing CMC and cellulose in a ratio of 1:1.

Purpose: Assessment of whether film formation is possible in the absence of pullulan.

### 7) Composition Including CMC, Cellulose, and Another Cellulose (HPMC) (1-7)

Composition: The composition consists only of CMC, cellulose, and HPMC, excluding pullulan. The composition was prepared by mixing CMC, cellulose and HPMC in a ratio of 1:1:1.

Purpose: Evaluation of the formability and physical properties of cellulose and HPMC-based films without pullulan.

Meanwhile, in the experiment of the present invention, the water dissociation time was measured to compare the dissolution rate of the film, and based on this, the solubility was defined as slowly dissolved (Δ), moderately dissolved (∘), and very rapidly dissolved (⊚).
⊚ (very rapidly dissolved): The film is completely dissolved in 10 seconds
∘ (moderately dissolved): The film is completely dissolved in 10 seconds or more and 30 seconds or less.
Δ (slowly dissolved): It takes 30 seconds or more, and some of the film remains even after a certain amount of time.
(Failure to dissolve or long-term persistence): The film does not completely dissolve even after 120 seconds or more.

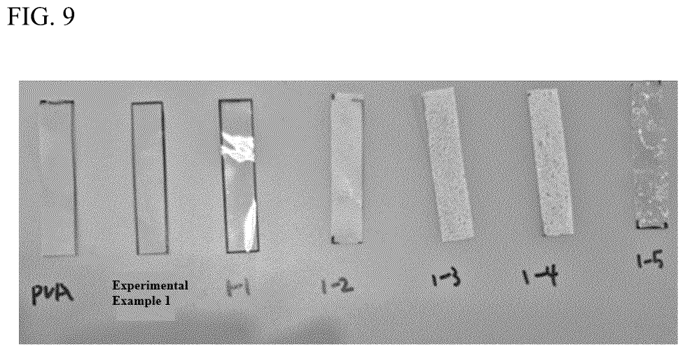

The actual films formed by each composition method are as shown in FIG. 9. Referring to FIG. 9, Comparative Example 1 (PVA-based film) had a transparent and uniform surface, and Experimental Example 1 (optimal composition of the present invention) also exhibited a relatively uniform film shape. Meanwhile, in Experimental Example 2, it was confirmed that the shape and surface properties of the film varied as the composition was changed, and in particular, in the composition including HPMC, the film surface tended to have a porous structure. This is a result reflecting the gelation properties of HPMC, suggesting that it can increase film structural stability in a certain composition.

### 3. Result Analysis

In order to evaluate the mechanical strength, viscosity, and water dissociation of the water-dissolvable film of the present invention, the physical properties were measured for Comparative Example 1 (PVA-based film), Experimental Example 1 (optimal composition of the present invention), and Experimental Example 2 (various variations of the composition), and the results are as follows.

TABLE 1

| | | Viscosity | Tensile | |
|---|---|---|---|---|
| | | (cps, Sp4, | strength | Water |
| Number | Composition | 20 rpm) | (N) | dissociation |
| Comparative | PVA | 2,710 | 44.80 | ⊚ |
| Example 1 | | | | |
| Experimental | pullulan + | 2,350 | 43.30 | ⊚ |
| Example 1 | CMC + | | | |
| | cellulose | | | |
| 1-1 | pullulan + | 2,030 | 14.31 | ◯ |
| | CMC | | | |
| 1-2 | pullulan + | 1,350 | 19.64 | ◯ |
| | cellulose | | | |
| 1-3 | pullulan + | 1,710 | 22.67 | Δ |
| | other | | | |
| | cellulose (HPMC) | | | |
| 1-4 | pullulan + | 6,100 | 29.29 | Δ |
| | CMC + other | | | |
| | cellulose (HPMC) | | | |
| 1-5 | pullulan + | 6,130 | 34.33 | Δ |
| | CMC + | | | |
| | cellulose + other | | | |
| | cellulose (HPMC) | | | |
| 1-6 | CMC + cellulose | 428 | Film | — |
| | | | formation | |
| | | | failure | |
| 1-7 | CMC + | 514 | Film | — |
| | cellulose + other | | formation | |
| | cellulose (HPMC) | | failure | |

### (1) Viscosity Analysis

As a result of the viscosity measurement, the viscosity of Comparative Example 1 (PVA-based film) was measured to be 2,710 cps, and the viscosity of Experimental Example 1 (optimal composition of the present invention) was confirmed to be 2,350 cps. Compared with the PVA-based solution, the mixed solution of the present invention had a slightly lower viscosity, but maintained a viscosity level sufficient for film formation.

In Experimental Example 2, the viscosity varied as the composition was changed, and the composition including pullulan and CMC (1-1) and the composition including only pullulan and cellulose (1-2) showed relatively low values of 2,030 cps and 1,350 cps. This suggests that the viscosity control effect is limited when CMC or cellulose is added alone. On the other hand, the viscosities of the composition including pullulan and another cellulose (HPMC) (1-3) and the composition including pullulan, CMC, and HPMC (1-4) were measured to be 1,710 cps and 6,100 cps, respectively, indicating that the viscosity change is large when HPMC is added. In particular, the composition including pullulan, CMC, cellulose, and HPMC (1-5) showed a very high viscosity of 6,130 cps, suggesting that the excessive increase in viscosity may cause difficulties in uniform application of the film.

For the composition including only CMC and cellulose (1-6) and the composition including only CMC, cellulose, and HPMC (1-7), the values were 428 cps and 514 cps, respectively, suggesting that pullulan plays an important role in film formation.

### (2) Tensile Strength Analysis

As a result of the tensile strength measurement, the tensile strength of Comparative Example 1 (PVA-based film) was measured to be 44.80 N, and the tensile strength of Experimental Example 1 (optimal composition of the present invention) was confirmed to be 43.30 N. This means that the film of the present invention can secure mechanical strength at a level similar to that of the PVA-based film, suggesting the possibility of replacing the existing PVA-based film.

In Experimental Example 2, the tensile strength values varied as the composition was changed, and the composition including only pullulan and CMC (1-1) showed a tensile strength of 14.31 N, and the composition including only pullulan and cellulose (1-2) showed a tensile strength of 19.64 N, indicating relatively low mechanical strength. This shows that the mechanical strength enhancement effect is limited when cellulose or CMC is added alone.

On the other hand, the tensile strength of the composition including pullulan and another cellulose (HPMC) (1-3) was confirmed to be 22.67 N, and the tensile strength of the composition including pullulan, CMC, and HPMC (1-4) was confirmed to be 29.29 N, and when HPMC was included, the mechanical strength tended to improve. In particular, the composition including pullulan, CMC, cellulose, and HPMC (1-5) showed the highest tensile strength of 34.33 N, but there is a possibility that viscosity increase and application uniformity problems may occur.

For the composition including only CMC and cellulose (1-6) and the composition including only CMC, cellulose, and HPMC (1-7), tensile strength measurement was not possible, indicating that pullulan plays an essential role in film formation and securing mechanical strength.

### (3) Water Dissociation Evaluation

As a result of the water dissociation evaluation, Comparative Example 1 (PVA-based film) was completely dissolved within 10 seconds (⊚), and Experimental Example 1 (optimal composition of the present invention) was also completely dissolved within 10 seconds (⊚), showing a dissolution rate similar to that of the PVA-based film.

In Experimental Example 2, the water dissociation varied as the composition was changed, and the composition including only pullulan and CMC (1-1) and the composition including only pullulan and cellulose (1-2) were completely dissolved in 10 seconds or more and 30 seconds or less (o), showing relatively fast solubility.

On the other hand, the composition including pullulan and another cellulose (HPMC) (1-3), the composition including pullulan, CMC, and HPMC (1-4), and the composition including pullulan, CMC, cellulose, and HPMC (1-5) took 30 seconds or more and showed a tendency for a certain amount of film to remain (A). This is interpreted as a decrease in the dissolution rate with an increase in the structural strength of the film when HPMC is included.

For the composition including only CMC and cellulose (1-6) and the composition including only CMC, cellulose, and HPMC (1-7), film formation failed, making solubility evaluation impossible.

### (4) Optimal Composition Derivation

As a result of comprehensively analyzing the above experiments, it was confirmed that Experimental Example 1 (composition including pullulan, CMC, and cellulose) achieved the optimal balance in terms of viscosity (2,350 cps), tensile strength (43.30 N), and water dissociation (⊚).

It was confirmed that this composition was more eco-friendly than the existing PVA while maintaining a mechanical strength similar to that of the PVA-based film, and the dissolution rate was also maintained at the same level as that of the existing PVA film.

In addition, referring to FIG. 10, a tensile strength-strain curve of a film manufactured with the optimal composition of the present invention is illustrated. As can be seen in the graph, the water-dissolvable film of the present invention was analyzed to exhibit a rapid increase in stress in the initial elastic section, exhibit a maximum tensile strength of about 20 MPa, and then maintain stress at a constant level before fracture occurs at a strain of about 70%.

Meanwhile, the tensile strength values in MPa are converted to suit the experimental conditions as follows. The cross-sectional area (width X thickness) of the specimen used in this experiment is 10 mm×0.21 mm=2.1 mm²=2.1×10⁻⁶ m². Therefore, 20 MPa (=20 N/mm²)×2.1 mm²=42 N, which means that the film of the present invention can withstand a maximum tensile load of about 42 N.

These characteristics exhibit a level similar to that of the mechanical properties of the PVA-based film, suggesting that the dissolution rate may be optimized while ensuring the durability and tensile strength of the film. In particular, the fact that the stress maintenance region after the elastic section is formed relatively widely indicates that the composition of the present invention is effective in maintaining the durability of the film, and it may be interpreted that the interaction between the pullulan, CMC, and cellulose increases the stability of the film structure.

As described above, it can be confirmed that the water-dissolvable film utilizing the pullulan-CMC-cellulose combination of the present invention has a mechanical strength equivalent to that of the existing PVA-based film, while being implemented using an environmentally friendly material. In particular, by utilizing pullulan as the main skeleton of the film, inducing viscosity control and homogeneous film formation through CMC, and adding cellulose as a reinforcing material, mechanical strength may be maintained.

In addition, the film of the present invention has a water dissociation rate similar to that of the PVA-based film and exhibits the characteristic of rapidly dissolving upon contact with water. This suggests the potential to replace existing PVA-based products in eco-friendly, water-dissolvable packaging and disposable film applications.

In addition, the film manufacturing process of the present invention has the advantage of not requiring a high-temperature process and being easy to combine with various auxiliary raw materials (moisturizers, functional additives, etc.).

For example, when a surfactant is additionally included in the composition (pullulan, carboxymethyl cellulose, and cellulose) according to an embodiment of the present invention, it may be utilized as a cosmetic sheet such as a cleansing sheet. The shape of a film manufactured through this composition may be formed as shown in FIG. 11, and may be configured to be suitable for cleansing and cosmetic applications by having a soft texture and appropriate water dissociation.

Through this, the film can be utilized in various industrial applications such as food packaging materials, medical films, cosmetic sheets, and functional dissolving films, and has value as an eco-friendly alternative material with excellent biodegradability and improved chemical stability compared to existing synthetic polymer films.

As a result, the water-dissolvable film based on the pullulan-CMC-cellulose of the present invention overcomes the limitations of the existing PVA-based film and provides an optimal composition that may simultaneously satisfy eco-friendliness, mechanical stability, and rapid solubility, thereby suggesting a new solution with high applicability in various industrial fields.

### Experimental Method and Result Analysis (Second Experiment)

In order to evaluate the mechanical properties of the water-dissolvable film based on the pullulan-CMC-cellulose of the present invention according to the relative ratio of the main components, tensile strength and viscosity measurement experiments were performed. Experiments in which CMC and cellulose were each included in amounts of 1 to 25% by weight relative to pullulan were performed. The experimental conditions and methods are the same as those of the first experiment, and the experimental results are as follows.

TABLE 2

| | | Tensile strength | Water |
|---|---|---|---|
| Number | Composition | (N) | dissociation |
| Experimental | CMC is 1% of pullulan | 21.21 | ◯ |
| Example 1 | and cellulose is 1% | | |
| | of pullulan | | |
| Experimental | CMC is 2% of pullulan | 35.80 | ⊚ |
| Example 2 | and cellulose is 2% | | |
| | of pullulan | | |
| Experimental | CMC is 5% of pullulan | 39.02 | ⊚ |
| Example 3 | and cellulose | | |
| | is 5% of pullulan | | |
| Experimental | CMC is 12.5% of | 43.30 | ⊚ |
| Example 4 | pullulan and | | |
| | cellulose is 12.5% | | |
| Experimental | of pullulan CMC is | 47.66 | ◯ |
| Example 5 | 20% of pullulan and | | |
| | cellulose is 20% | | |
| | of pullulan | | |
| Experimental | CMC is 22% of pullulan | 49.90 | Δ |
| Example 6 | and cellulose is 22% | | |
| | of pullulan | | |

It can be confirmed that the water dissociation decreases slightly when cellulose and CMC reach 20%, and becomes very poor when they reach 22%. This is analyzed as being due to the fact that the strength becomes very strong as the amount of cellulose increases and because much gelation occurs as the amount of CMC increases. In addition, in the case of Experimental Example 1 where cellulose and CMC are 1%, it can be confirmed that the tensile strength is drastically reduced compared to that of Experimental Example 2. When the tensile strength falls below 25 N, a problem occurs where it becomes difficult to maintain the film form.

As a result, it can be seen that when CMC and cellulose are each blended in an amount of 2 to 20% relative to pullulan, the film has an appropriate tensile strength for water dissociation and film formation.

Although an embodiment of the present invention has been described above with reference to the accompanying drawings, those of ordinary skill in the art to which the present invention pertains should understand that the present invention may be embodied in other specific forms without changing the technical spirit or essential features thereof. Therefore, it should be understood that the embodiments described above are illustrative in all respects and not restrictive.

The specific implementations described in the present invention are only exemplary and do not limit the scope of the present invention in any way. For the sake of brevity of the specification, descriptions of conventional electronic components, control systems, software, and other functional aspects of the systems may be omitted. In addition, the connections or lines between components depicted in the drawings are merely illustrative of functional connections and/or physical or circuit connections, and may be represented as various functional connections, physical connections, or circuit connections that are replaceable or additional in an actual device. In addition, when there is no specific mention such as “essential,” “importantly,” etc., it may not be a component absolutely necessary for the application of the present invention.

It is understood that the specific order or hierarchy of steps in the presented processes are examples of exemplary approaches. It is to be understood that, based on design priorities, the specific order or hierarchy of steps in the processes within the scope of the present invention may be rearranged. The attached method claims provide elements of various steps in a sample order, but are not meant to be limited to the specific order or hierarchy of steps presented.

The description of the presented embodiments is provided to enable those skilled in the art to use or practice the invention. Various modifications to these embodiments will be apparent to those skilled in the art, and the general principles defined herein may be applied to other embodiments without departing from the scope of the invention. Accordingly, the present invention is not intended to be limited to the embodiments presented herein, but should be construed in the broadest scope consistent with the principles and novel features disclosed herein.

### MODES OF THE INVENTION

In the best mode for carrying out the invention as described above, related contents have been described.

1. A water-dissolvable film comprising cellulose,
wherein the water-dissolvable film includes pullulan, carboxymethyl cellulose (CMC), and cellulose.

2. The water-dissolvable film of claim 1, wherein the water-dissolvable film includes 2 parts by weight or more and 20 parts by weight or less of the carboxymethyl cellulose based on 100 parts by weight of the pullulan, and includes 2 parts by weight or more and 20 parts by weight or less of the cellulose based on 100 parts by weight of the pullulan.

3. The water-dissolvable film of claim 1, wherein the water-dissolvable film is formed using a mixed solution having a viscosity in a range of 1,500 centipoise (cps) or more and 6,500 centipoise or less.

4. The water-dissolvable film of claim 1, wherein the water-dissolvable film has a tensile strength in a range of 20 N or more and 50 N or less.

5. The water-dissolvable film of claim 1, wherein the water-dissolvable film forms an intermolecular hydrogen bonding network between the pullulan and the carboxymethyl cellulose.

6. The water-dissolvable film of claim 1, wherein the water-dissolvable film includes main raw materials including the pullulan, the carboxymethyl cellulose, and the cellulose and an auxiliary raw material for controlling the mechanical strength, flexibility, and dissolution characteristics of the film, and the auxiliary raw material is included in an amount of 100 parts by weight or less based on 100 parts by weight of the main raw materials.

7. The water-dissolvable film of claim 6, wherein the auxiliary raw material includes at least one of carrageenan, cyclodextrin, sorbitol, and glycerin.

8. The water-dissolvable film of claim 1, wherein the water-dissolvable film includes an additional raw material including a surfactant, in which case the film has a final viscosity in a range of 15,000 centipoise or more and 30,000 centipoise or less.

9. A method of manufacturing a water-dissolvable film including cellulose, comprising:
preparing a mixed solution including pullulan, carboxymethyl cellulose, and cellulose;
applying the mixed solution onto a flat plate; and
drying the applied mixed solution to form a water-dissolvable film.
